(19) United States
(12) Patent Application Publication
(10) Pub. No.: US 2020/0402672 A1
(43) Pub. Date: Dec. 24, 2020

(54) SYSTEMS AND METHODS TO GROUP RELATED MEDICAL RESULTS DERIVED FROM A CORPUS OF MEDICAL LITERATURE

(71) Applicant: Evid Science, Inc., El Segundo, CA (US)

(72) Inventors: Matthew Michelson, La Canada, CA (US); Michael Ross, Los Angeles, CA (US)

(21) Appl. No.: 16/448,413

(22) Filed: Jun. 21, 2019

(51) Int. Cl. G16H 70/20; G16H 50/70; G16H 50/20

(52) U.S. Cl. CPC G16H 70/20; G16H 70/60; G16H 50/20; G16H 50/70

(57) ABSTRACT

Systems and methods to group related medical results derived from a corpus of medical literature are disclosed. Exemplary implementations may: obtain a set of medical results derived from a corpus of medical literature, the set of medical results being represented by attribute values of attributes; determine, for individual ones of the attribute values, individual sets of feature values for grouping features; determine, based on the individual sets of feature values, groups of the attribute values; and/or perform other operations.

### FIELD OF THE DISCLOSURE

The present disclosure relates to systems and methods to group related medical results derived from a corpus of medical literature.

### BACKGROUND

Techniques are known that generate a repository of medical results based on a corpus of medical literature, which may also be refreshed as new literature is published. The medical literature may include, for example, studies, research papers, randomized controlled trials, and/or other literature. Techniques may utilize artificial intelligence systems to extract details from the literature and represent the extracted information in a structured form providing access to the medical results by both machines and people. By automatically extracting outcomes data, sometimes called results or evidence, from the medical literature, such a structured data set of results/evidence may be created at a scale covering a multitude of the published literature.

Medical ontologies may refer to the representation and/or organization of medical terminologies. Numerous medical ontologies exist. Examples include RxNorm, MeSH, SNOMED CT, ICD-9, I CD-10, and CPT. RxNorm is a standardized nomenclature for clinical drugs and drug delivery devices. MeSH is a list of vocabulary terms used for subject analysis of biomedical literature at the National Library of Medicine. SNOMED CT is a comprehensive and granular clinical terminology developed by the College of American Pathologists and often utilized in Electronic Health Records. ICD-9 is a medical classification of diseases and their associated signs and symptoms—often used to assign diagnostic and procedure codes. CPT is a procedural terminology from the American Medical Association used to track inpatient and outpatient medical procedures.

### SUMMARY

One aspect of the present disclosure relates to a system configured to group related medical results derived from a corpus of medical literature. As described above, techniques are known to derive structured representations of medical data from a corpus of text. However, there has yet to be a way to understand which of the results should be grouped together in meaningful ways, beyond what may be derived from known medical ontologies. For instance, “Tylenol” and “acetaminophen” may refer to the same drug, “AMD” and “age-related macular degeneration” may refer to the same disease, “Crohn″s disease” is a specific type of “Inflammatory Bowel Disease” (e.g., via ontology mappings), and one may choose to group “remission at 5 days”, “remission at 14 weeks” and “clinical remission” together, although one may not, as well.

One or more implementations of the systems and methods presented herein may utilize feature values of grouping features of medical results in order to group the medical results. The systems and methods may utilize machine-learning and/or other artificial intelligence which may cause the groupings to evolve over time providing insightful relationships which may go beyond what may be gleaned from medical literature alone, including existing ontologies. The grouping features may include one or more of synonyms, semantic relatedness (e.g., embedding based statistics or ontology mappings), acronyms, “word shortenings” (e.g., “BUD” for budesonide), and/or other grouping features described herein. In some implementations, multiple grouping features may be combined to create new ontologies describing medical concepts and/or how they are related. By way of non-limiting illustration, deriving structured representation of medical results from a corpus of medical literature via artificial intelligence may extract two therapies used in medical studies: “Tylenol, taken as a weekly injection of 1000 mg” and “acetaminophen, two 500 mg pills, twice a day.” An ontology derived from one or more implementations of the systems and methods presented herein may represent that each therapy uses the same drug, but different dosages, schedules, and/or delivery methods. Similarly, an extracted outcome like “nausea, two weeks after treatment” may be represented within the ontology as a sub-type of other outcome concepts such as “adverse event” or “adverse event at 14 days.”

The feature values of individual grouping features and/or combinations of grouping features (e.g., a derived ontology) may be used to create on-the-fly groupings relevant to a particular purpose. For instance, when examining a collection of papers for a particular disease, papers may be grouped by drug, so that drug outcomes may be compared. In some instances, papers may be grouped by drug and dosage, so that outcomes of each dosage may be compared. Such groupings may be auto-extracted and then optionally modified by human users for display and analysis. Derived ontologies may provide the ability to create rich flexible groupings. Without such groupings, it may be harder to draw stronger conclusions from the derived medical results. For instance, by grouping together multiple results about Parkinson's or “budesonide” (independent of dosage) or “remission,” the system may build stronger inferences versus analyzing individual results. Further, visualizations and summaries may utilize the grouped medical results. One or more of these features may help users derive insights about comparative performance of therapies, understand the landscape of options for a given disease, and/or develop evidence-based decisions.

One or more implementations of a system configured to group related medical results may include one or more hardware processors configured by machine-readable instructions. The processor(s) may be configured to obtain a set of medical results derived from a corpus of medical literature. The set of medical results may be represented by attribute values of attributes. By way of non-limiting illustration, a first result may be represented by a first attribute value, a second result being represented by a second attribute value, and/or other results may be represented by other attribute values.

The processor(s) may be configured to determine, for individual ones of the attribute values, individual sets of feature values for individual grouping features. By way of non-limiting illustration, a first set of feature values for a first grouping feature and/or other sets of feature values for other grouping features may be determined for the first attribute value. By way of non-limiting illustration, a second set of feature values for the first grouping feature and/or other sets of feature values for other grouping features may be determined for the second attribute value.

The processor(s) may be configured to determine, based on sets of feature values for the individual attribute values, groups of the attribute values. By way of non-limiting illustration, a first group including the first attribute value and the second attribute value may be determined based on one or more of the first set of feature values, the second set of feature values, and/or other information.

As used herein, any association (or relation, or reflection, or indication, or correspondency) involving servers, processors, client computing platforms, and/or another entity or object that interacts with any part of the system and/or plays a part in the operation of the system, may be a one-to-one association, a one-to-many association, a many-to-one association, and/or a many-to-many association or N-to-M association (note that N and M may be different numbers greater than 1).

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured to group related medical results derived from a corpus of medical literature, in accordance with one or more implementations.

FIG. 2 illustrates a method to group related medical results derived from a corpus of medical literature, in accordance with one or more implementations.

FIG. 3 illustrates a visual depiction of groupings of attribute values, in accordance with one or more implementations.

FIG. 4 illustrates a user interface, in accordance with one or more implementations.

### DETAILED DESCRIPTION

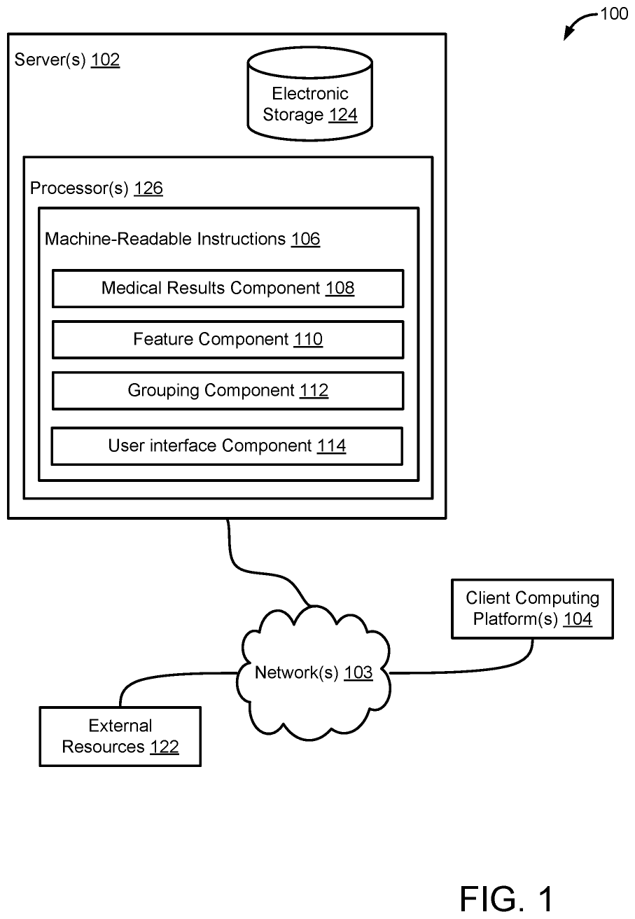

FIG. 1 illustrates a system 100 configured to group related medical results derived from a corpus of medical literature, in accordance with one or more implementations. Feature values of individual grouping features and/or combinations of grouping features may be used to create on-the-fly groupings relevant to a particular purpose. For instance, when examining a collection of papers for a particular disease, papers may be grouped by drug, so that drug outcomes may be compared. In some instances, papers may be grouped by drug and dosage, so that outcomes of each dosage may be compared. Such groupings may be extracted and then optionally modified by human users for display and analysis. Derived ontologies may provide the ability to create rich flexible groupings. Without such groupings, it may be harder to draw stronger conclusions from the derived medical results. In some implementations, the relationships derived from the systems and methods herein may create new medical ontologies.

In some implementations, system 100 may include one or more of one or more servers 102, one or more client computing platforms 104, external resources 122, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via individual ones of the client computing platform(s) 104.

Server(s) 102 may be configured by machine-readable instructions 106. Machine-readable instructions 106 may include one or more instruction components. Executing the machine-readable instructions 106 may cause server(s) 102 to facilitate grouping related medical results derived from a corpus of medical literature. The instruction components may include computer program components. The instruction components may include one or more of medical results component 108, feature component 110, grouping component 112, user interface component 114, and/or other instruction components.

The medical results component 108 may be configured to obtain a set of medical results derived from a corpus of medical literature. The medical results may have been derived from a corpus of medical literature via artificial intelligence and/or other techniques. The set of medical results may be stored in a structured database. The set of medical results may be structured by virtue of the medical results being represented by attribute values of pre-defined and/or derived attributes. The attributes may include one or more of an intervention attribute, a group attribute, an outcome attribute, a problem attribute, a context attribute, and/or other attributes.

An attribute value of an intervention attribute may specify intervention information. Intervention information may be related to the medical intervention being implemented. The intervention information may include one or more of a drug used, an amount of a drug administered, a time period over which a drug was administered (e.g., also referred to as “measurement-time”), a procedure performed, and/or other information.

An attribute value of a group attribute may specify patient-level information and/or other information. The patient-level information may include information about the patients and/or a patient group. The patient-level information may include one or more of age, age range, demographic, inclusion and/or exclusion study criteria, sex, and/or other information including information determined during the study (e.g., the patients who experienced a particular side effect of a drug, and/or other information).

An attribute value of an outcome attribute may specify outcome information. The outcome information may include one or more of a result of treatment, a time period in which a certain result was obtained, and/or other information. By way of non-limiting illustration, a result of treatment may include one or more of an adverse event, remission, hospitalization, and/or other information.

An attribute value of a problem attribute may specify problem information. The problem information may be related to what is being treated. Problem information may include one or more of a disease present, a physical injury present, disease study population criteria, and/or other information. By way of non-limiting illustration, a disease present may include Crohn's disease and/or other disease. By way of non-limiting illustration, a physical injury present may include a bone fracture and/or other physical problem.

An attribute value of a context attribute may specify information derived either directly and/or indirectly from the medical literature from which the medical results were derived. Directly derived may refer to information present on the face of the medical literature. Indirectly derived may refer to information that may be systematically and/or logically determined from the information present on the face of the medical literature. By way of non-limiting illustration, an attribute value of a context attribute may include one or more of a citation to medical literature, a link to the literature, relative risk (e.g., a ratio of the probability of an event occurring in the exposed group versus the probability of the event occurring in the non-exposed group), a classification of the medical literature (e.g., random control study, clinical trial, etc.), a generated sentence, and/or other information. A sentence may be generated by taking discrete information (e.g., individual words, numbers, and/or other content) and generating a natural language sentence.

It is noted that the above descriptions of various attributes having attribute values defining medical results within a database are for illustrative purposes only and not to be considered limiting. Instead, those skilled in the art may recognize other attribute values of these attributes and/or other attributes which may be used to define the stored data.

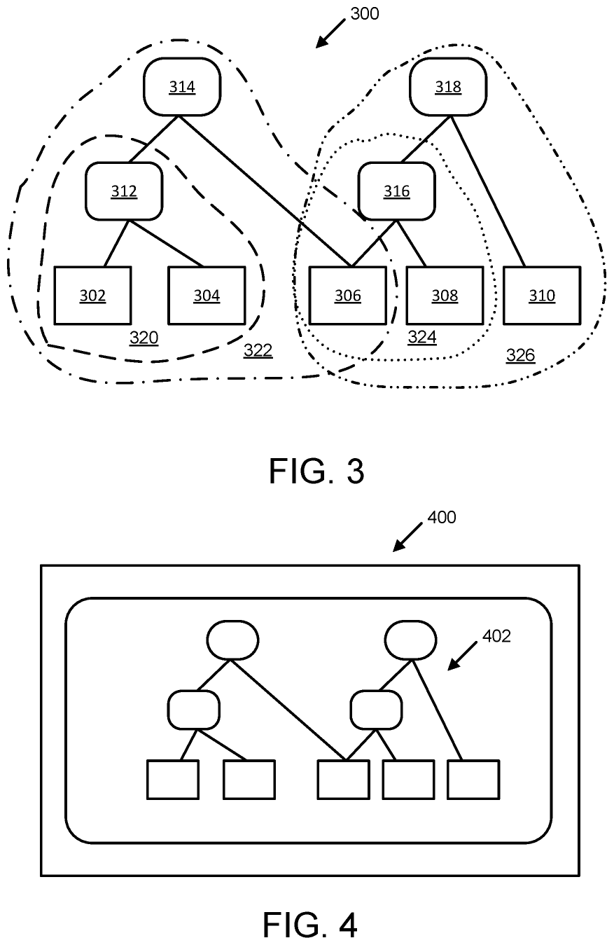

Feature component 110 may be configured to determine, for individual ones of the attribute values, individual sets of feature values for individual grouping features. For an individual attribute value, an individual set of feature values may be determined for an individual grouping feature. There may be multiple grouping features. Therefore, for an individual attribute value, feature component 110 may determine multiple sets of feature values for multiple grouping features, where an individual set of feature values may correspond to an individual grouping feature. In some implementations, the feature values for grouping features of the individual attribute values may facilitate determining relationships between individual ones of the attribute values in order to group the attribute values. In some implementations, feature values of multiple grouping features may result in robust groupings of attribute values. For example, attribute values may be grouped based on feature values of multiple different grouping features. The utilization of feature values of multiple different grouping features may cause a given attribute value to be assigned into more than one group (see, e.g., grouping component 112 and/or visualization in FIG. 3). The grouping of attribute values in more than one group may provide more insight into the set of medical results than other methods of grouping medical results and/or medical concepts. The assignment of individual attribute values into more than one group may further create new ontologies (see, e.g., the visualization in FIG. 3).

In some implementations, the grouping features may include one or more of one or more implementation features, one or more linguistic features, one or more source features, and/or other grouping features.

A feature value of an implementation feature of a given attribute value may include additional information and/or detail of the given attribute value. The additional information and/or detail about the given attribute value may include additional medical results related to the attribute value. In some implementations, the additional medical results related to the attribute value may include other attribute values of the same attribute. By way of non-limiting illustration, if an attribute value of an intervention attribute specifies a drug used, a feature value of an implementation feature may specify one or more of an amount of the drug used, a time period over which a drug was administered, and/or other information.

In some implementations, an implementation feature may include a type-of feature. A feature value of a type-of feature for an attribute value may specify a “generic” name of the attribute value. A generic name may include a layman's term, other generally accepted medical terminologies and/or classifications (e.g., as determined by existing medical ontologies and/or other sources), and/or other information. By way of non-limiting illustration, an attribute may comprise a problem attribute, the attribute value may specify a physical injury present such as a “facture,” a feature value of a type-of feature for “fracture” may specify that “fracture” is a “type-of” “break”. By way of non-limiting illustration, an attribute may comprise a problem attribute, the attribute value may specify a disease present such as a “Crohn's disease,” a feature value of a type-of feature for “Crohn's disease” may specify that “Crohn's disease” is a “type-of” “Inflammatory Bowel Disease” (with “Inflammatory Bowel Disease” being a generally accepted medical classification of Crohn's disease).

The one or more linguistic features may be related to language-based analysis of the attribute values. The one or more linguistic features may include one or more of a synonym feature, an antonym feature, an ontology feature, a word-embedding feature, a stemming and lemmatization feature, an abbreviation feature, a sub-word feature, an n-gram feature, and/or other features.

A feature value of a synonym feature of an attribute value may specify a synonym of the individual attribute value. Synonym may refer to words or expressions of the same language that may have the same or nearly the same meaning. A set of feature values of the synonym feature may specify a set of synonyms of the attribute value. By way of non-limiting illustration, “Amyotrophic Lateral Sclerosis” may be a synonym of “Lou Gehrig's Disease.” By way of non-limiting illustration, one or more of “incidence left atrial appendage thrombus”, “left atrial appendage thrombus present”, “left atrial thrombus”, “presence left atrial appendage thrombus” and/or other phrases may be synonymous.

A feature value of an antonym feature of an individual attribute value may specify an antonym of the individual attribute value. Antonym may refer to words or expressions of opposite meaning. By way of non-limiting illustration, “survival” may be an antonym of “mortality.”

A feature value of an ontology feature of an individual attribute value may specify information related to the individual attribute value derived from one or more existing medical ontologies. By way of non-limiting illustration, the information related to the individual attribute value derived from one or more existing medical ontologies may include relationships between the individual attribute value and one or more medical concepts and/or terminologies as specified in the one or more existing medical ontologies. By way of non-limiting illustration, an existing ontology may describe that “survival” may be antonymous with “mortality.”

A feature value of a word-embedding feature of an individual attribute value may specify a representation of the individual attribute value as determined through one or more word-embedding models. The word-embedding models may include language modeling and/or feature learning techniques where words or phrases may be mapped to vectors of real numbers. The word-embedding models may include one or more of Tomas Mikolov's Word2vec, Stanford University's GloVe, AllenNLP's Elmo, Google's BERT, and/or other techniques.

A feature value of a stemming and lemmatization feature of an individual attribute value may specify stems and/or lemma of individual attribute value. A stem may refer to a part of a word such as a root or derivational morphemes. A lemma may refer to inflectional endings of words. Feature values of the stemming and lemmatization feature may be determined by one or more of a production technique, suffix-stripping algorithms, additional algorithm criteria, Lemmatization algorithms, n-gram analysis, affix stemmers, matching algorithms, and/or other techniques.

A feature value of an abbreviation feature of an individual attribute value may specify an abbreviation formed from the initial letters of the attribute value and/or an expansion the abbreviated form of the attribute value.

A feature value of a sub-word feature of an individual attribute value may specify a sequence of characters within a string of characters of the attribute value. The sequence of characters may themselves form another word. The sequence of characters may be contiguous within the string of characters or noncontiguous characters within the string of characters.

A feature value of an n-gram feature of an individual attribute value may specify a contiguous sequence of “n” items from the individual attribute value. The items may include one or more of phonemes, syllables, letters, base pairs, and/or other information. A feature value of an n-gram feature may be determined through one or more n-gram models.

The one or more source features may be related to information that may be derived from, and/or may be present on the face of, individual medical literature and/or set of medical literature in the corpus of medical literature. By way of non-limiting illustration, the one or more source features may include one or more of a frequency count feature, co-occurrence feature, syntax feature, and/or other features. A feature value of a count feature of an attribute value may specify a frequency count of the occurrence of the attribute value within the corpus. A feature value of a co-occurrence feature of an attribute value may quantify the co-occurrence of the attribute value with other words. A feature value of a syntax feature of an attribute value may specify one or more arrangements of words and phrases in which the attribute value is present in the medical literature of the corpus.

The determination of feature values for the grouping features may be accomplished through one or more techniques. The techniques may include one or more of machine-learning, database searching, and/or other techniques.

The machine-learning techniques may include specially trained machine-learning algorithms configured to output feature values. A machine-learning technique may include one or more of a natural language processing technique, clustering, sequence labeling, and/or other techniques.

A machine-learning approach to natural language processing may return feature values related to one or more of synonyms, antonyms, word-embedding, n-grams, stemming or lemmatizing, acronyms, and/or other feature values. By way of non-limiting illustration, natural language processing techniques to determine stemming and lemmatization feature values may include one or more of a production technique, suffix-stripping algorithms, additional algorithm criteria, Lemmatization algorithms, n-gram analysis, affix stemmers, matching algorithms, and/or other techniques.

The clustering may utilize a model configured to group a set of feature values in such a way that the feature values in the same group (called a cluster) may be more similar to each other than to those in other groups (clusters). Feature values determined to be in the same cluster may also mean the feature values may be representative of a common grouping feature. In some implementations, clustering may be accomplished by one or more of hierarchical clustering, centroid-based clustering, distribution-based clustering, density-based clustering, and/or other techniques. By way of non-limiting illustration, a clustering model may determine that the grouping feature of “synonym feature” for the attribute value of “left atrial appendage thrombus” may include the following set of feature values: “incidence left atrial appendage thrombus”, “left atrial appendage thrombus present”, “left atrial thrombus”, “presence left atrial appendage thrombus”, and/or other feature values

By way of non-limiting illustration, a clustering model may determine that the grouping feature of “synonym feature” for the attribute value of “Lipid lowering” may include the following set of feature values: “lipid-lowering response”, “month 12: lipid lowering”, “month 4: anti-hypertensive and lipid lowering”, “month 4: anti-hypertensive and/or lipid lowering”, “month 4: lipid lowering”, and/or other feature values.

One or more machine-learning techniques may require training, referred to as supervised learning. Training may be accomplished through user input, while the learning aspect of the machine-learning techniques may occur over time as the system continues to run and/or more literature is added to the corpus. For example, a user may provide a machine-learning model with training data. The training data may include exemplars that specify sets of feature values for individual attribute values. The exemplars may be determined by a human and thereafter used to train a model. It is contemplated that other types of a machine learning, such as unsupervised learning, may also be utilized within the scope of this disclosure.

Database searching may include querying one or more literature sources. The literature sources may include one or more of one or more existing medical ontologies, one or more dictionaries, one or more thesauruses, and/or other sources. Using existing ontologies and/or literature sources may facilitate resolving feature values such as one or more of synonym, antonyms, acronyms, and/or other values. In some implementations, the literature sources may be stored locally, e.g., within electronic storage 124. In some implementations, the literature sources may comprise one or more of the external resources 122 and accessible over network(s) 103. In some implementations, the queries may be web-based queries. By way of non-limiting illustration, feature component 110 may be configured to transmit requests (e.g., queries) for information from one or more sources. The information may be generated as results returned by one or more of Structured Query Language (SQL), pictorials, graphs, complex results, and/or other representations of the requests.

By way of non-limiting illustration, feature component 110 may be configured to determine one or more of a first set of feature values for a first grouping features for the first attribute value, a second set of feature values for the first grouping feature for the second attribute value, and/or other feature values for the first attribute value, second attribute value, and/or other attribute values.

Grouping component 112 may be configured to determine, based on the sets of feature values for the attribute values, groups of the attribute values. In some implementations, a group of attribute values may include attribute values which commonly share one or more feature values of one or more grouping features. In some implementations, grouping component 112 may be configured to determine which attribute values share one or more common feature values. The grouping component 112 may be configured to assign attribute values determined to share one or more common feature values to a group. In some implementations, an individual attribute value may be assigned to more than one group. In some implementations, commonly sharing one or more feature values may include one or more of being an exact match, sharing one or more words and/or phrases, and/or other considerations of common sharing of feature values.

In some implementations, the grouping component 112 may be configured to assign names to individual groups. In some implementations, an individual name of an individual group may comprise words and/or phrases representative of the one or more commonly shared feature values associated with the individual group, and/or may include other information. In some implementations, a word and/or phrase representing of the one or more commonly shared feature values may comprise one or more of a summary of the one or more commonly shared feature values, a short-hand description of the one or more commonly shared features values, at least one of the one or more commonly shared feature values, and/or other information. By way of non-limiting illustration, a summary may comprise natural language description of the commonly shared feature values. By way of non-limiting illustration, a short-hand description may comprise a word and/or phrase that encompasses the commonly shared feature values in a short form. For example, values of “serious adverse events” and “reported adverse events” may be assigned to a group named “adverse events.”

By way of non-limiting illustration, grouping component 112 may be configured to determine a first group including the first attribute value and the second attribute value based on the first set of feature values and the second set of feature values. The first group may be determined based on the first set of feature values and the second set of feature values sharing one or more feature values.

User interface component 114 may be configured to present a user interface. The user interface may be configured to facilitate one or more of user editing of one or more groupings determined by grouping component 112, presentation of one or more visualizations utilizing the groupings determined by grouping component 112, and/or other functionality. A user interface may include one or more user interface elements configured to facilitate user interaction with the user interface. User interface elements may include one or more of text input fields, drop down menus, check boxes, display windows, virtual buttons, and/or other elements configured to facilitate user interaction. In some implementations, a visualization may include one or more of a graph, a chart, a heat map, a table, a plot, and/or other visualization utilizing the groupings. By way of non-limiting illustration, a visualization may include one or more of a comparison table, a bar chart showing how the intervention performed, an odds ratio plot visualization, a timeline of how interventions change over time, and/or other visualizations.

FIG. 3 illustrates a visualization of groupings of attribute values, in accordance with one or more implementations. The attribute values to be grouped may include one or more of a first attribute value 302, a second attribute value 304, a third attribute value 306, a fourth attribute value 308, a fifth attribute value 310, and/or other attribute values. For individual ones of the attribute values, individual sets of feature values for individual grouping features may be determined.

In FIG. 3, a first set of feature values for a first grouping feature may be determined for the first attribute value 302. The first set of feature values may include a first feature value 312 and/or other feature values. A second set of feature values for the first grouping feature may be determined for the second attribute value 304. The second set of feature values may include the first feature value 312 and/or other feature values. Based on sets of feature values for the individual attribute values, groups of the attribute values may be determined. For example, a first group 320 (indicated by the dashed line) including the first attribute value 302 and the second attribute value 304 may be determined based on the first set of feature values and the second set of feature values commonly sharing the first feature value 312.

In FIG. 3, a third set of feature values for a second grouping feature may be determined for the first attribute value 302. The third set of feature values may include a second feature value 314 and/or other feature values. A fourth set of feature values for the second grouping feature may be determined for the second attribute value 304. The second set of feature values may include second feature value 314 and/or other feature values. A fifth set of feature values for the second grouping feature may be determined for the third attribute value 306. The fifth set of feature values may include the second feature value 314 and/or other feature values. Based on sets of feature values for the individual attribute values, groups of the attribute values may be determined. For example, a second group 322 (indicated by the dash-dot line) including the first attribute value 302, the second attribute value 304, and the third attribute value 306 may be determined based on the third set of feature values, the fourth set of feature values, and the fifth set of feature values commonly sharing the second feature value 314.

By way of non-limiting illustration, a sixth set of feature values for a third grouping feature may be determined for the third attribute value 306. The sixth set of feature values may include a third feature value 316 and/or other feature values. A seventh set of feature values for the third grouping feature may be determined for the fourth attribute value 308. The seventh set of sixth values may include the third feature value 316 and/or other feature values. Based on sets of feature values for the individual attribute values, groups of the attribute values may be determined. For example, a third group 324 (indicated by the dotted line) including the third attribute value 306 and the fourth attribute value 308 may be determined based on the sixth set of feature values and the seventh set of feature values commonly sharing the third feature value 316.

In FIG. 3, an eighth set of feature values for a fourth grouping feature may be determined for the third attribute value 306. The eighth set of feature values may include a fourth feature value 318 and/or other feature values. A ninth set of feature values for the fourth grouping feature may be determined for the fourth attribute value 308. The ninth set of feature values may include fourth feature value 318 and/or other feature values. A tenth set of feature values for the fourth grouping feature may be determined for the fifth attribute value 310. The tenth set of feature values may include the fourth feature value 318 and/or other feature values. Based on sets of feature values for the individual attribute values, groups of the attribute values may be determined. For example, a fourth group 326 (indicated by the dash-dot-dot line) including the third attribute value 306, the fourth attribute value 308, and the fifth attribute value 310 may be determined based on the eighth set of feature values, the ninth set of feature values, and the tenth set of feature values commonly sharing the fourth feature value 318.

As a practical example of the visualization in FIG. 3, consider the first attribute value 302 to be a value for an outcome attribute which specifies “discontinuation because of adverse events after 14 days” and the second attribute value 304 to be a value of an outcome attribute which specifies “left the study after the second week.” A feature value for a grouping feature comprising an implementation feature for the first attribute value 302 may be “time: 14 days.” A feature value for a grouping feature comprising an implementation feature for the second attribute value 304 may be “time: 14 days.” As such, the first feature value 312 of “time: 14 days” may be commonly shared between the first attribute value 302 and the second attribute value 304 causing them to be assigned to the first group 320.

FIG. 4 illustrates a user interface 400, in accordance with one or more implementations. The user interface 400 may be configured to facilitate one or more of user editing of one or more groupings, presentation of one or more visualizations utilizing the groupings, and/or other functionality. In the figure, an example of facilitating user editing of one or more groupings is shown. By way of non-limiting illustration, the user interface 400 may show a visualization of groupings 402 of attribute values determined in accordance with one or more implementations of the systems and methods presented herein. For illustrative purposes, a set of groupings similar to that shown in FIG. 3 is depicted. The user interface 400 may facilitate editing of the groupings 402. Editing may include one or more of adding and/or removing attribute values (e.g., represented by the square ikons) to and/or from a given group, changing names assigned to a given group, and/or other editing. Adding and/or removing attribute values to and/or from a given group may be facilitated by one or more of drag-and-drop input, text input via a text input element (not shown in FIG. 4) and/or other inputs. For example, a menu (not shown in FIG. 4) may be included on the user interface 400 showing a listing of attribute values that may be selected and added to the groupings 402 shown in the user interface 400. In some implementations, groupings may be changed based on modifying the connecting lines between icons (e.g., square icons and/or rounded corner icons).

Returning to FIG. 1, in some implementations, server(s) 102, client computing platform(s) 104, and/or external resources 122 may be operatively linked via one or more electronic communication links. For example, such electronic communication links may be established, at least in part, via network(s) 103 such as the Internet and/or other networks. It will be appreciated that this is not intended to be limiting, and that the scope of this disclosure includes implementations in which server(s) 102, client computing platform(s) 104, and/or external resources 122 may be operatively linked via some other communication media.

An individual client computing platform of one or more client computing platforms 104 may include one or more processors configured to execute computer program components. The computer program components may be configured to enable an expert or user associated with the individual client computing platform to interface with system 100 and/or external resources 122, and/or provide other functionality attributed herein to client computing platform(s) 104. By way of non-limiting example, the individual client computing platform may include one or more of a desktop computer, a laptop computer, a handheld computer, a tablet computing platform, a NetBook, a Smartphone, a gaming console, and/or other computing platforms.

External resources 122 may include sources of information outside of system 100, external entities participating with system 100, and/or other resources. In some implementations, some or all of the functionality attributed herein to external resources 122 may be provided by resources included in system 100.

Server(s) 102 may include electronic storage 124, one or more processors 126, and/or other components. Server(s) 102 may include communication lines, or ports to enable the exchange of information with network(s) 103 and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 124 may comprise non-transitory storage media that electronically stores information. The electronic storage media of electronic storage 124 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 124 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 124 may include one or more virtual storage resources (e.g., cloud storage, a virtual private network, and/or other virtual storage resources). Electronic storage 124 may store software algorithms, information determined by processor(s) 126, information received from server(s) 102, information received from client computing platform(s) 104, and/or other information that enables server(s) 102 to function as described herein.

Processor(s) 126 may be configured to provide information processing capabilities in server(s) 102. As such, processor(s) 126 may include one or more of a digital processor, an analog processor, a digital circuit designed to process information, an analog circuit designed to process information, a state machine, and/or other mechanisms for electronically processing information. Although processor(s) 126 is shown in FIG. 1 as a single entity, this is for illustrative purposes only. In some implementations, processor(s) 126 may include a plurality of processing units. These processing units may be physically located within the same device, or processor(s) 126 may represent processing functionality of a plurality of devices operating in coordination. Processor(s) 126 may be configured to execute components 108, 110, 112, and/or 114, and/or other components. Processor(s) 126 may be configured to execute components 108, 110, 112, and/or 114, and/or other components by software; hardware; firmware; some combination of software, hardware, and/or firmware; and/or other mechanisms for configuring processing capabilities on processor(s) 126. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, and/or 114 are illustrated in FIG. 1 as being implemented within a single processing unit, in implementations in which processor(s) 126 includes multiple processing units, one or more of components 108, 110, 112, and/or 114 may be implemented remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other ones of components 108, 110, 112, and/or 114. As another example, processor(s) 126 may be configured to execute one or more additional components that may perform some or all of the functionality attributed below to one of components 108, 110, 112, and/or 114.

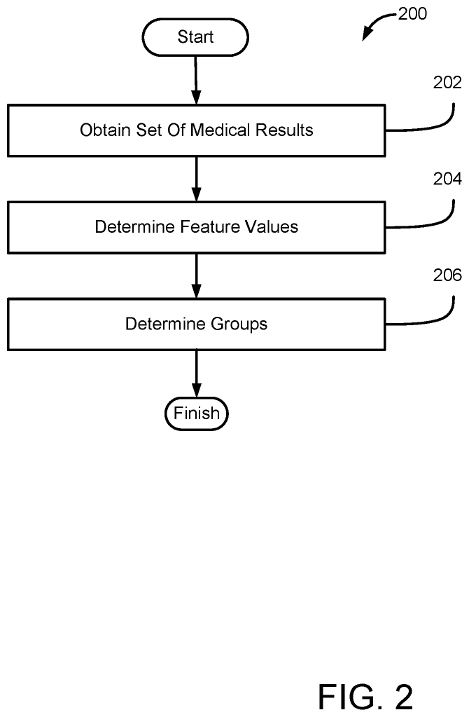

FIG. 2 illustrates a method 200 to group related medical results derived from a corpus of medical literature, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include obtaining a set of medical results derived from a corpus of medical literature. The set of medical results may be represented by attribute values of attributes. Operation 202 may be performed by one or more hardware processors configured by machine-readable instructions including a component that is the same as or similar to medical results component 108, in accordance with one or more implementations.

An operation 204 may include determining, for individual ones of the attribute values, individual sets of feature values for individual grouping features. Operation 204 may be performed by one or more hardware processors configured by machine-readable instructions including a component that is the same as or similar to feature component 110, in accordance with one or more implementations.

An operation 206 may include determining, based on sets of feature values for the attribute values, groups of the attribute values. Operation 206 may be performed by one or more hardware processors configured by machine-readable instructions including a component that is the same as or similar to grouping component 112, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured to group related medical results derived from a corpus of medical literature, the system comprising:
one or more physical processors configured by machine-readable instructions to:
obtain a set of medical results derived from a corpus of medical literature, the set of medical results being represented by attribute values of attributes, a first result being represented by a first attribute value, and a second result being represented by a second attribute value;
determine, for individual ones of the attribute values, individual sets of feature values for individual grouping features, such that a first set of feature values for a first grouping feature is determined for the first attribute value, and a second set of feature values for the first grouping feature is determined for the second attribute value; and
determine, based on sets of feature values for the individual attribute values, groups of the attribute values, such that a first group including the first attribute value and the second attribute value is determined based on the first set of feature values and the second set of feature values.

2. The system of claim 1, wherein determining the groups of the attribute values comprises determining which attribute values commonly share one or more feature values, such that the first group is determined based on the first set of feature values and the second set of feature values sharing one or more feature values.

3. The system of claim 2, wherein a third result is represented by a third attribute value, a third set of feature values for the first grouping feature is determined for the third attribute value, and wherein a second group including the first attribute value and the third attribute value is determined based on the first set of feature values and the third set of feature values sharing one or more feature values.

4. The system of claim 1, wherein the grouping features include one or more of one or more implementation features, one or more linguistic features, and/or one or more source features.

5. The system of claim 4, wherein a feature value of an implementation feature of a given attribute value includes additional detail about the given attribute value.

6. The system of claim 4, wherein the one or more linguistic features include one or more of a synonym feature, an antonym feature, an ontology feature, a word-embedding feature, a stemming and lemmatization feature, an abbreviation feature, a sub-word feature, or an n-gram feature.

7. The system of claim 4, wherein the one or more source features include one or more of a frequency count feature, a co-occurrence feature, or a syntax feature.

8. The system of claim 1, wherein determining the feature values for the grouping features is based on one or both of machine-learning or database searching.

9. The system of claim 8, wherein the database searching includes querying one or more literature sources, the one or more literature sources including one or more of one or more existing medical ontologies, one or more dictionaries, or one or more thesauruses.

10. The system of claim 8, wherein the machine-learning comprises supervised learning trained based on user-provided training data.

11. A method to group related medical results derived from a corpus of medical literature, the method comprising:
obtaining a set of medical results derived from a corpus of medical literature, the set of medical results being represented by attribute values of attributes, a first result being represented by a first attribute value, and a second result being represented by a second attribute value;
determining, for individual ones of the attribute values, individual sets of feature values for individual grouping features, such that a first set of feature values for a first grouping feature is determined for the first attribute value, and a second set of feature values for the first grouping feature is determined for the second attribute value; and
determining, based on sets of feature values for the individual attribute values, groups of the attribute values, such that a first group including the first attribute value and the second attribute value is determined based on the first set of feature values and the second set of feature values.

12. The method of claim 11, wherein determining the groups of the attribute values comprises determining which attribute values commonly share one or more feature values, such that the first group is determined based on the first set of feature values and the second set of feature values sharing one or more feature values.

13. The method of claim 12, wherein a third result is represented by a third attribute value, a third set of feature values for the first grouping feature is determined for the third attribute value, and wherein a second group including the first attribute value and the third attribute value is determined based on the first set of feature values and the third set of feature values sharing one or more feature values.

14. The method of claim 11, wherein the grouping features include one or more of one or more implementation features, one or more linguistic features, and/or one or more source features.

15. The method of claim 14, wherein a feature value of an implementation feature of a given attribute value includes additional detail about the given attribute value.

16. The method of claim 14, wherein the one or more linguistic features include one or more of a synonym feature, an antonym feature, an ontology feature, a word-embedding feature, a stemming and lemmatization feature, an abbreviation feature, a sub-word feature, or an n-gram feature.

17. The method of claim 14, wherein the one or more source features include one or more of a frequency count feature, a co-occurrence feature, or a syntax feature.

18. The method of claim 11, wherein determining the feature values for the grouping features is based on one or both of machine-learning or database searching.

19. The method of claim 18, wherein the database searching includes querying one or more literature sources, the one or more literature sources including one or more of one or more existing medical ontologies, one or more dictionaries, or one or more thesauruses.

20. The method of claim 18, wherein the machine-learning comprises supervised learning trained based on user-provided training data.
